(19) United States
(12) Patent Application Publication
(10) Pub. No.: US 2007/0191838 A1
(43) Pub. Date: Aug. 16, 2007

(54) Interspinous devices and methods of use

(75) Inventors: Aurelien Bruneau, Memphis, TN (US); Eric C. Lange, Collierville, TN (US); Randall Noel Allard, Germantown, TN (US); Kent M. Anderson, Memphis, TN (US)

Correspondence Address: COATS & BENNETT, PLLC, 1400 Crescent Green, Suite 300, Cary, NC, 27518, US

(73) Assignee: SDGI Holdings, Inc.

(21) Appl. No.: 11/341,233

(22) Filed: Jan. 27, 2006

(51) Int. Cl. A61F 2/30

(52) U.S. Cl. 606/61

(57) ABSTRACT

The present application is directed to devices and methods for spacing apart spinous processes. In one embodiment, the device includes a body having a central portion that separates first and second sections. The body is constructed of a combination of an elastic material and a bio-absorbable material. When initially implanted within a patient, the bio-absorbable material affects the stiffness of the device. The device stiffness changes to that of the elastic material as the bio-absorbable material is absorbed by the body.

### BACKGROUND

The present application is directed to devices and methods for stabilizing vertebral members, and more particularly, to interspinous devices that are positioned between the spinous processes of vertebral members.

Vertebral members comprise a body, pedicles, laminae, and processes. The body has an hourglass shape with a thinner middle section and wider ends. Intervertebral discs are positioned between the bodies of adjacent vertebral members to permit flexion, extension, lateral bending, and rotation. The pedicles are two short rounded members that extend posteriorly from the body, and the laminae are two flattened members that extend medially from the pedicles. The processes are projections that serve as insertion points for the ligaments and tendons. The processes include the articular processes, transverse processes, and the spinous process. The spinous process is a single member that extends posteriorly from the junction of the two lamina. The spinous process acts as a lever to effect motion of the vertebral member.

Various conditions may lead to damage of the intervertebral discs and/or the vertebral members. The damage may result from a variety of causes including a specific event such as trauma, a degenerative condition, a tumor, or infection. Damage to the intervertebral discs and vertebral members can lead to pain, neurological deficit, and/or loss of motion.

One method of correcting the damage is insertion of a device between the spinous processes of adjacent vertebral members. The device may reduce or eliminate the pain and neurological deficit, and increase the range of motion.

### SUMMARY

The present application is directed to devices and methods for spacing spinous processes. In one embodiment, the device includes a body having a central portion that separates first and second sections. The body is constructed of a combination of an elastic material and a bio-absorbable material. When initially implanted within a patient, the bio-absorbable material affects the stiffness of the device. The stiffness changes to that of the elastic material as the bio-absorbable material is absorbed by the body.

### BRIEF DESCRIPTION OF THE DRAWINGS

FIG. 1 is a cross section view illustrating a device according to one embodiment.

FIG. 2 is a side view illustrating a device positioned between spinous processes according to one embodiment.

FIG. 3 is a cross section view illustrating a device according to one embodiment.

FIG. 4 is a cross section view illustrating a device according to one embodiment.

FIG. 5 is a cross section view illustrating a device according to one embodiment.

FIG. 6 is a cross section view illustrating a device according to one embodiment.

FIG. 7A is a cross section view illustrating a device in a first orientation according to one embodiment.

FIG. 7B is a schematic side view illustrating the device of FIG. 7A in a second orientation according to one embodiment.

FIG. 8 is a cross section view illustrating a device according to one embodiment.

FIG. 9 is a perspective view illustrating a band extending around a central portion of a device according to one embodiment.

### DETAILED DESCRIPTION

The present application is directed to devices and methods for spacing spinous processes of vertebral members. In one embodiment, the device includes a body having a central portion that separates first and second sections. The body is constructed of a combination of an elastic material and a bio-absorbable material. When initially implanted within a patient, the bio-absorbable material affects the stiffness of the device. The stiffness of the device changes to that of the elastic material as the bio-absorbable material is absorbed by the body. The change may be gradual, or may be sudden.

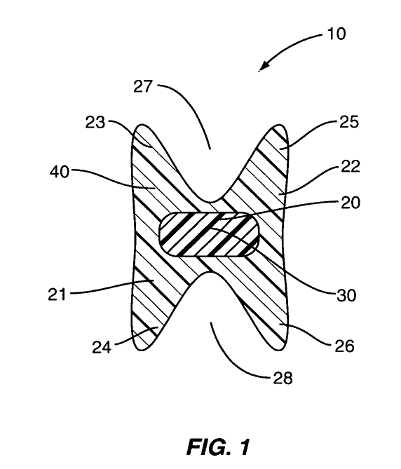

FIG. 1 illustrates one embodiment of the device 10 including a central portion 20 positioned between first and second sections 21, 22. In this embodiment, first section 21 includes elongated arms 23, 24 and second section 22 includes elongated arms 25, 26. Gaps 27, 28 may be formed between the first and second sections 21, 22 to receive the spinous processes.

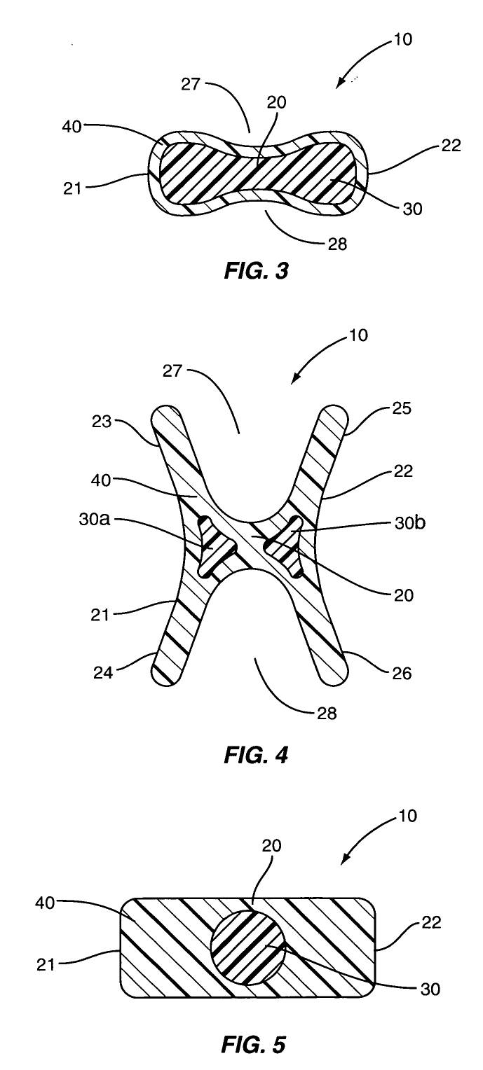

FIG. 3 illustrates another embodiment of a device 10. This embodiment features less pronounced first and second sections 21, 22 that are separated by a central portion 20. Gaps 27, 28 formed between the sections 21, 22 are smaller and less well defined. In another embodiment, not illustrated, the device 10 has no definite shape prior to insertion between the spinous processes 102. The device 10 conforms to the shape of the interspinous space between the spinous processes. It is understood that various embodiments of the device 10 may have a variety of shapes and sizes. The central portion 21 and the first and second sections 21, 22 may have different sizes and configurations depending upon the context of use. In one embodiment as illustrated in FIGS. 1 and 3, the first and second sections 21, 22 are substantially symmetrical relative to the central portion 20. In another embodiment, sections 21, 22 are asymmetrical.

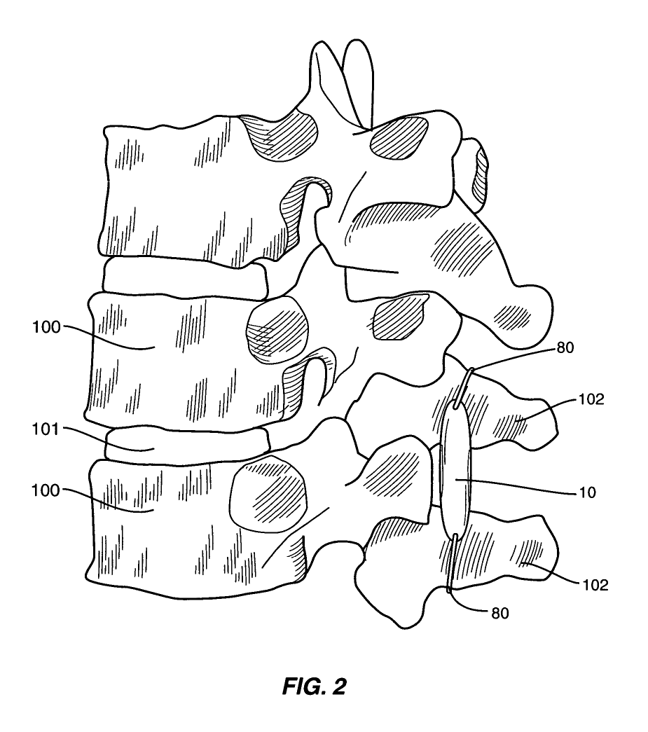

FIG. 2 illustrates one embodiment of a device 10 mounted between spinous processes 102. The gaps 27, 28 are sized to receive the spinous process 102 of each vertebral member 100. Tethers 80 may be used to maintain the device 10 positioned within the interspinous space. In one embodiment, tethers 80 are bio-absorbable and are absorbed by the body after a period of time.

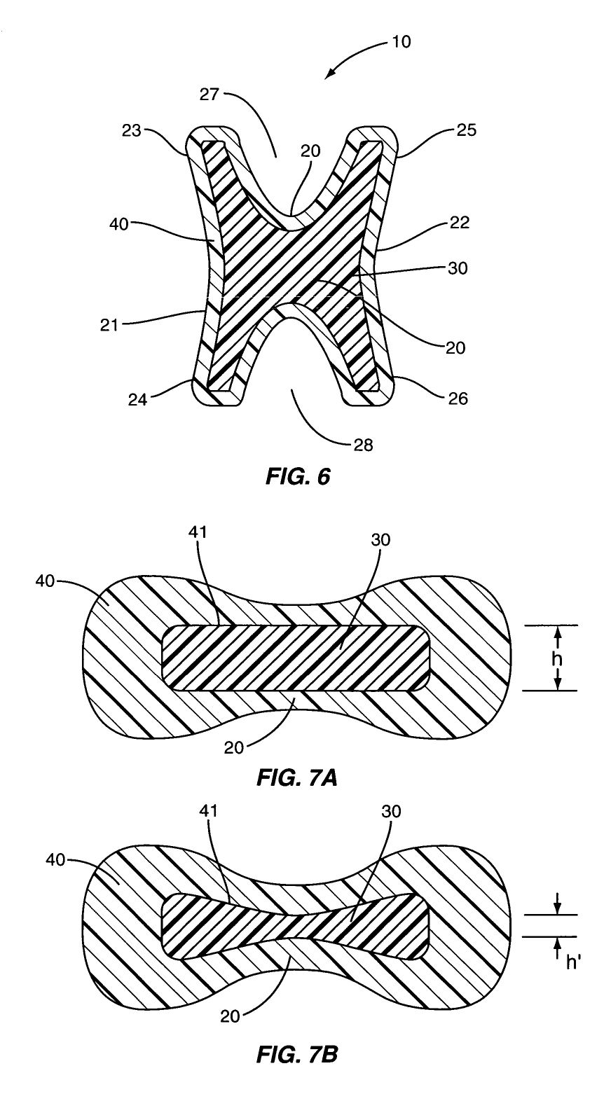

The device 10 is partially constructed from an elastic member 30. In one embodiment, the elastic member 30 is positioned within the central portion 20 of the device 10. This positioning provides for the elastic member 30 to be positioned within the interspinous space. The elastic member 30 may extend through the entirety or a limited amount of the central portion 20. FIG. 1 illustrates one embodiment of the elastic member 30 positioned within the central portion 20. In this embodiment, member 30 is comprised of a single unit that extends through the entirety of the central portion 20 and into the each of the first and second sections 21, 22. FIG. 4 illustrates an embodiment comprising two separate elastic members 30a, 30b each positioned partially within the central portion 20. First elastic member 30a extends partially into the central portion 20 from a first lateral side, and second elastic member 30b extends partially into the central portion 20 from a second lateral side. FIG. 5 illustrates another embodiment with the elastic member 30 entirely within the central portion 20. FIG. 6 illustrates an embodiment where the elastic member 30 comprises a majority of the central portion 20, and first and second sections 21, 22.

The height and width of the elastic member 30 may be adequate for spacing apart the spinous processes 102 and may vary depending upon the context. The elastic member 30 of FIG. 1 has a substantially constant height and width throughout. In the embodiment of FIG. 3, the outer ends of the elastic member 30 include greater heights than a middle section. Elastic member 30 may include a symmetrical or asymmetrical shape about the central portion 20.

The elastic member 30 may be formed for a wide variety of biocompatible polymeric materials, including elastic materials, such as elastomeric materials, hydrogels or other hydrophilic-polymers, or composites thereof. Suitable elastomers include silicone, polyurethane, copolymers of silicone and polyurethane, polyolefins, such as polyisobutylene and polyisoprene, neoprene, nitrile, vulcanized rubber and combinations thereof. Suitable hydrogels include natural hydrogels, and those formed from polyvinyl alcohol, acrylamides such as polyacrylic acid and poly(acrylonitrile-acrylic acid), polyurethanes, polyethylene glycol, poly(N-vinyl-2-pyrrolidone), acrylates such as poly(2-hydroxy ethyl methacrylate) and copolymers of acrylates with N-vinyl pyrrolidone, N-vinyl lactams, acrylamide, polyurethanes and polyacrylonitrile, or may be other similar materials that form a hydrogel. The hydrogel materials may further be cross-linked to provide further strength to the implant. Examples of polyurethanes include thermoplastic polyurethanes, aliphatic polyurethanes, segmented polyurethanes, hydrophilic polyurethanes, polyether-urethane, polycarbonate-urethane and silicone polyether-urethane. Other suitable hydrophilic polymers include naturally-occurring materials such as glucomannan gel, hyaluronic acid, polysaccharides, such as cross-linked carboxyl-containing polysaccharides, and combinations thereof.

The nature of the materials employed to form the elastic member 30 may be selected so the devices 10 include sufficient stiffness to space apart the spinous processes 102. The term stiffness is used to refer to the resistance of an elastic body to deflection by an applied force.

Device 10 is also constructed from a bio-absorbable material 40. Bio-absorbable material 40 may position the elastic material 30 within the interspinous space and/or affect the stiffness of the device 10. Bio-absorbable material 40 provides the positioning and/or stiffness functions for a limited time after the device 10 is implanted as the material 40 is eventually absorbed by the body. In one embodiment, the bio-absorbable material 40 is gradually absorbed by the body. During this initial period, the body may heal to an extent that the elastic material 30 is adequate to support the vertebral members 100 and/or the body is able to position the elastic material 30. In one embodiment, the bio-absorbable material 40 is replaced with tissue, such as fibrous tissue and fibrous scar tissue that aids in positioning the elastic material 30 permanently within the interspinous space.

Bio-absorbable material 40 may be formed from a wide variety of natural or synthetic materials. In one embodiment, the material 40 is elastic or elastomeric. In one embodiment, material 40 is deformable. In one embodiment, material 40 is non-compliant. Suitable bio-absorbable materials 40 fibrin, albumin, collagen, elastin, silk and other proteins, polyethylene oxide, cyanoacrylate, polylactic acid, polyester, polyglycolic acid, polypropylene fumarate, tyrosine-based polycarbonate and combinations thereof. Other suitable materials include demineralized bone matrix. In one embodiment, bio-absorbable material 40 may be a woven fabric.

Bio-absorbable material 40 may function to position the elastic material 30 within the interspinous space. In embodiments as illustrated in FIGS. 1 and 4, bio-absorbable material 40 forms the arms 23, 24, 25, 26 that position the elastic material 30 within the interspinous space. Similarly, the embodiments of FIGS. 3 and 5 include bio-absorbable material 40 forming the outer areas of the first and second sections 21, 22. It is understood that the bio-absorbable material 40 may be formed into a variety of different shapes and sizes for positioning the elastic material 30.

Bio-absorbable material 40 may function to affect an overall stiffness of the device 10. In one embodiment, the bio-absorbable material 40 and elastic material 30 work in combination to support the vertebral members 100 when the device 10 is initially implanted within the body. Over time, the bio-absorbable material 40 is absorbed by the body and the stiffness lessens or changes resulting in the elastic material 30 providing an increasing amount of the support characteristics of the overall device 10. In one embodiment, the bio-absorbable material 40 is completely absorbed by the body with only the elastic material 30 remaining.

In one embodiment, the bio-absorbable material 40 has a high stiffness. During an initial period, the bio-absorbable material 40 alone or substantially supports the vertebral members 100 and prevents and/or restricts movement. After the bio-absorbable material 40 is absorbed by the body, the elastic material 30 provides the support characteristics of the device 10 and provides for movement of the vertebral members 100. In one embodiment, a high stiffness bio-absorbable material 40 is used when a vertebral member 100 and/or disc 101 have been damaged and motion within the spinal area is prevented. After the vertebral member 100 and/or disc 101 have healed, the bio-sbsorbable material 40 has been absorbed and the elastic material 30 includes a stiffness to provide for motion within the spinal area. In one embodiment, the bio-absorbable material 40 slowly absorbs into the body and its stiffness gradually lessens. During this period, the overall stiffness properties of the device 10 are shared by both materials 30, 40.

In one embodiment, both materials 30, 40 are elastic with the bio-absorbable material 40 having a greater initial stiffness. Initially, the overall stiffness of the device 10 is defined by a combination of the materials 30, 40 with the stiffer bio-absorbable material 40 providing a greater amount to the overall stiffness. As the bio-absorbable material 40 becomes absorbed by the body it provides a lessening amount to the overall stiffness. In one embodiment, the overall stiffness of the device 10 decreases as the bio-absorbable material 40 is absorbed by the body thus allowing for a greater range of motion of the vertebral members 100. Eventually, the bio-absorbable material 40 is completely absorbed by the body and the elastic member 30 alone provides support to the vertebral members 100, and may provide for an even greater range of motion.

Bio-absorbable material 40 may also affect the stiffness properties by confining the deformation of the elastic material 30. In one embodiment, the bio-sbsorbable material prevents and/or restricts the deformation of the elastic material 30 during movement of the vertebral members 100. One embodiment of this confinement is illustrated in FIGS. 7A and 7B with the bio-absorbable material 40 including a holding section 41 in which the elastic material 30 is positioned. In a first orientation as illustrated in FIG. 7A, the holding section 41 includes a first shape and a first height h. During movement of the vertebral members 100 such as during extension, the bio-absorbable material 40 is deformed causing the holding section 41 to change shape and decrease in height to h′. The elastic material 30 is likewise deformed during the movement, but the shape is confined to conform to the shape of the holding section 41. The elastic material 30 may exhibit a variable stiffness during the confined deformation and the stiffness may increase upon additional deformation from the first orientation. In one embodiment, the holding section 41 is completely contained within the bio-absorbable material 40. In another embodiment, the holding section 41 includes an opening to an exterior of the device 10.

In one embodiment, the stiffness of the bio-absorbable material 40 decreases as the material is absorbed by the body. In one embodiment, this decreased stiffness results in less confinement of the elastic material 30 causing a lower overall stiffness of the device 10.

In one embodiment, the bio-absorbable material 40 is pliable and non-cmopliant. An embodiment may include the bio-absorbable material 40 being constructed from polyester. In another embodiment, the bio-absorbable material 40 is a woven fabric. In one embodiment, the bio-absorbable material 40 itself has no stiffness properties. However, the bio-absorbable material 40 confines the elastic material 30 and thus affects the overall stiffness properties of the device 10.

In one embodiment, the elastic material 30 is connected to the bio-absorbable material 40. The elastic material 30 may include a variety of features on an outer surface, including chemical modifications and surface configurations, that improve the bonding between outer surface of the elastic material 30 and a surface of the holding section 41. In one embodiment, the outer surface is chemically modified, such as by surface grafting, and pre-coating with a primer such as a layer of adhesive, sealant, or other like materials. The elastic material 30 may also include surface configurations such as macro-surface patterns or protuberances.

In one embodiment, the elastic material 30 fills the entirety of the holding section 41. In another embodiment, the holding section 41 has a volume greater than the elastic material 30. In one embodiment, the elastic material 30 is freely positioned within the holding section 41.

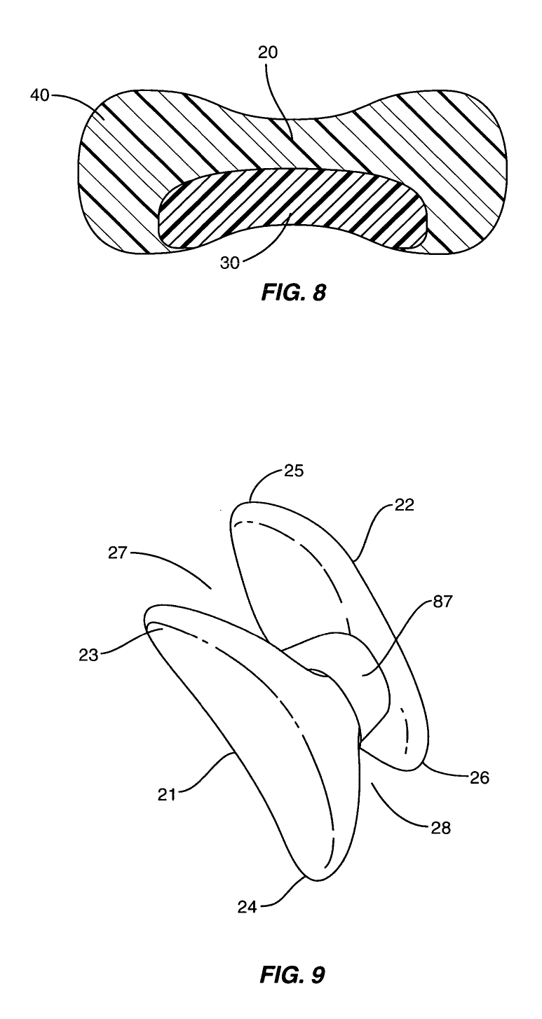

In one embodiment, bio-absorbable material 40 surrounds the entirety of the elastic material 30. Examples of this are illustrated in the embodiments of FIGS. 1, and 3-7. In another embodiment, bio-absorbable material 40 surrounds a limited section of the elastic material 30. FIG. 8 illustrates one embodiment with the bio-absorbable material 40 surrounding less than the entirety of the elastic material 30.

In one embodiment as illustrated in FIG. 9, a band 87 constructed of a bio-absorbable material 40 may extend around a section of the device 10. Band 90 may be constructed of the same or a different bio-absorbable material than material 40. In the embodiment of FIG. 9, the band 87 is positioned between the first and second sections 21, 22 and around the central portion 20. In another embodiment, band 87 extends around the first and second sections 21, 22, and the central portion 20. In one embodiment, band 87 prevents deformation of the elastic material 30 and/or the bio-absorbable material 40 thereby increasing an overall stiffness of the device 10. Band 87 may be constructed from a bio-absorbable material and begin to break down after an initial period thereby causing the overall stiffness of the device to change.

In one embodiment, the band 87 is attached to an exterior of the device 10. In another embodiment, band 87 is positioned completely or partially within the bio-absorbable material 40. Band 87 may extend around a portion or entirety of the elastic material 30. In one embodiment, multiple bands 87 extend around the elastic material 30. The multiple bands 87 may have the same or different support properties.

In another embodiment, bio-absorbable sutures or cables stabilize the device 10 during an initial period. The sutures and/or cables increase the overall stiffness of the device 10. As these begin to be absorbed by the body, the overall stiffness increases. In one embodiment, absorption of the sutures and/or cables results in a gradual change in the overall stiffness of the device 10. In one embodiment, the sutures and/or cables completely fail after a period of time resulting in a sudden and significant change in the overall stiffness.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A device to space first and second spinous processes comprising:
an elastic material positioned within a central portion that is positioned between the first and second spinous processes; and
a bio-absorbable material surrounding the elastic material;
the elastic material and bio-absorbable material acting in combination during an initial time period and comprising a first stiffness to space the first and second spinous processes, and comprising a second stiffness property after the initial time period to space the first and second spinous processes.

2. The device of claim 1, wherein the second stiffness is provided exclusively by the elastic material.

3. The device of claim 1, wherein the elastic material comprises first and second sections each at least partially positioned within the central portion.

4. The device of claim 1, wherein the bio-absorbable material comprises first and second sections positioned on lateral sides of the elastic material, the first and second sections comprising a greater height than the central portion.

5. The device of claim 4, further comprising a first set of arms extending upwardly from the first and second sections and a second set of arms extending downwardly from the first and second sections, the first and second sets of arms forming gaps to receive the spinous processes.

6. The device of claim 1, wherein the bio-absorbable material comprises a holding section to contain the elastic material.

7. The device of claim 1, further comprising a band positioned around the elastic material to control deformation of the elastic material during the initial time period.

8. A device to space first and second spinous processes within a body, the device comprising:
an elastic material positioned within a central portion between the first and second spinous processes; and
a bio-absorbable material surrounding the elastic material, the bio-absorbable material being absorbed into the body after an initial time period of being implanted within the body;
the elastic material and bio-absorbable material together providing a stiffness during the initial time period, and the elastic material alone providing the stiffness after the initial time period.

9. The device of claim 8, wherein the stiffness decreases during the initial time period.

10. The device of claim 9, wherein the stiffness is substantially constant after the initial time period.

11. The device of claim 8, wherein the bio-absorbable material provides a majority of the stiffness during a first part of the initial time period and the elastic material provides the majority of the stiffness during a second part of the initial time period.

12. The device of claim 8, wherein the bio-absorbable material is non-compliant.

13. The device of claim 8, wherein the bio-absorbable material is rigid during a first part of the initial time period.

14. The device of claim 8, wherein the bio-absorbable material and the elastic material are both deformable during the initial time period.

15. The device of claim 8, wherein the bio-absorbable material is constructed of a woven fabric.

16. The device of claim 8, wherein the bio-absorbable material completely surrounds the elastic material.

17. The device of claim 8, further comprising a band that extends around the elastic material, the band configured to restrict deformation of the elastic material.

18. A device to space first and second spinous processes within a body, the device comprising:
a bio-absorbable material comprising a holding section positioned within a central portion between the first and second spinous processes, the holding section varying between a first shape when the first and second spinous processes are in a first position and a second shape when the first and second spinous processes are in a second position; and
an elastic material confined within the holding section and having a shape determined by the holding section, the elastic material having a first stiffness when the holding section has a first shape, and a second stiffness when the holding section has a second shape;
a stiffness of the device being defined by the bio-absorbable material and the elastic material during an initial time period, and defined by the elastic material after the initial time period.

19. The device of claim 18, wherein the holding section is contained completely within the bio-absorbable material.

20. The device of claim 18, wherein the stiffness decreases during the initial time period.

21. The device of claim 18, wherein the stiffness is substantially constant after the initial time period.

22. The device of claim 18, wherein the bio-absorbable material provides a majority of the stiffness during a first part of the initial time period and the elastic material provides the majority of the stiffness during a second part of the initial time period.

23. A device to space first and second spinous processes comprising:
an elastic material;
a bio-absorbable material surrounding at least a portion of the elastic material, the elastic material and the bio-absorbable material comprising a shape to position the elastic material between the first and second spinous processes;
the elastic material and bio-absorbable material acting in combination during an initial time period and comprising a first stiffness to space the first and second spinous processes, and comprising a second stiffness property after the initial time period to space the first and second spinous processes.
